(19) United States
(12) Patent Application Publication
(10) Pub. No.: US 2017/0303788 A1
(43) Pub. Date: Oct. 26, 2017

(54) WEARABLE DEVICE FOR TISSUE MONITORING WITH EFFECTIVE AMBIENT LIGHT BLOCKING

(71) Applicant: PERFORMANCE ATHLYTICS, Austin, TX (US)

(72) Inventors: Paulo E. XAVIER DA SILVEIRA, Boulder, CO (US); Nithin O. RAJAN, Austin, TX (US); Dustin M. FRECKLETON, Austin, TX (US)

(73) Assignee: PERFORMANCE ATHLYTICS, Austin, TX (US)

(21) Appl. No.: 15/497,101

(22) Filed: Apr. 25, 2017

Related U.S. Application Data
(60) Provisional application No. 62/327,223, filed on Apr. 25, 2016.

(51) Int. Cl. A61B 5/00; A61B 5/1455; A61B 5/026; A61B 5/024; A61B 5/0205

(52) U.S. Cl. CPC A61B 5/0059; A61B 5/6828; A61B 5/6824; A61B 5/6801; A61B 5/0205; A61B 5/0261; A61B 5/02427; A61B 5/02416; A61B 5/1455

(57) ABSTRACT

Device configured to non-invasively perform tissue monitoring. The device can include at least two light emitting components and at least one photodiode configured to receive reflected light generated from the at least two light emitting components. The at least one photodiode is spaced apart from the at least two light emitting components. The device can include an analog-to-digital converter component configured to generate a digitized detected light signal based on data received from the at least one photodiode. The device can further include a processor configured to execute instructions to process the digitized signals. The device can included an ambient light blocking component configured to surround the at least two light emitting components and the at least one photodiode to prevent external light from entering a region bounded by the ambient light blocking component. The device can also include at least one filter mounted over the at least one photodiode.

### CROSS REFERENCE TO RELATED APPLICATIONS

This application claims priority to U.S. Provisional Application No. 62/327,223, filed Apr. 25, 2016, the contents of each are entirely incorporated by reference herein.

### FIELD

The present disclosure generally relates to a non-invasive tissue-monitoring device.

### BACKGROUND

There are several devices that have been created for non-invasive tissue-monitoring. These devices are constructed to emit light toward a user and measure the reflected light. For example, one such device is a pulse oximeter that can be used at a doctor's office or a hospital. The pulse oximeter uses emitted light to determine a user's pulse and blood oxygenation.

### BRIEF DESCRIPTION OF THE DRAWINGS

FIG. 1 is a schematic diagram of a non-invasive optical electronic device according to an example of this disclosure.

FIG. 2A is a schematic diagram of the front of a non-invasive optical-electronic device according to an example of this disclosure.

FIG. 2B is a schematic diagram of the back of a non-invasive optical-electronic device according to an example of this disclosure.

FIG. 2C is a schematic diagram of a spatially-resolved near infrared spectroscopy (NIRS) sensor that is included on a non-invasive optical-electronic device according to an example of the disclosure.

FIG. 3 illustrates the components of an optical-electronic device according to an example of this disclosure.

FIG. 4 is a plot of the extinction coefficients of deoxyhemoglobin and oxyhemoglobin as a function of wavelength over the visible range of wavelengths.

FIG. 5 is a plot of the ratio of the deoxyhemoglobin and oxyhemoglobin extinction coefficients as a function of wavelength over the visible range of wavelengths.

FIG. 6 illustrates an environment within which the non-invasive optical-electronic device can be implemented, according to an example of this disclosure.

FIG. 7 is a plot showing the absorption of light in the tissue of a wrist across various wavelengths.

FIG. 8 is a plot showing the photocurrent generated in a device of this disclosure by ambient light.

FIG. 9 is a plot showing the photocurrent generated by ambient light when the device is surrounded by the fabric composition of this disclosure.

FIG. 10 is a plot of solar irradiance taking into account photodetector (PD) responsivity and plastic window transmission.

FIG. 11 is a chart showing the light absorption properties of fabric compositions of this disclosure.

FIG. 12 is a plot showing the effect of an infrared (IR) cut-off filter.

### DETAILED DESCRIPTION

Various examples of the disclosure are discussed in detail below. While specific implementations are discussed, it should be understood that this is done for illustration purposes only. The other components and configurations can be used without parting from the spirit and scope of the disclosure.

It should be understood at the outset that although illustrative implementations of one or more examples are illustrated below, the disclosed device can be implemented using any number of techniques. The disclosure should in no way be limited to the illustrative implementations, drawings, and techniques illustrated herein, but can be modified within the scope of the appended claims along with their full scope of equivalents.

Unless otherwise specified, any use of any form of the terms “connect,” “engage,” “couple,” “attach,” or any other term describing an interaction between elements is not meant to limit the interaction to direct interaction between the elements and can also include indirect interaction between the elements described. In the following discussion and in the claims, the terms “including” and “comprising” are used in an open-ended fashion, and thus should be interpreted to mean “including, but not limited to . . . ”. The various characteristics described in more detail below, will be readily apparent to those skilled in the art with the aid of this disclosure upon reading the following detailed description, and by referring to the accompanying drawings.

The present disclosure generally relates to a device configured to non-invasively perform tissue monitoring. The device includes at least two light emitting components, at least one photodiode, an analog-to-digital converter component, a processor, an ambient light blocking component, and at least one filter mounted over the at least one photodiode. The photodiode is configured to receive reflected light generated from the at least two light emitting components, such that the at least one photodiode is spaced apart from the at least two light emitting components. The analog-to-digital converter is configured to generate a digitized detected light signal based on data received from the at least one photodiode. The processor is configured to execute instructions to process the digitized signals. The ambient light blocking component is configured to surround the at least two light emitting components and the at least one photodiode to prevent external light from entering a region bounded by the ambient light blocking component.

In an example, the device includes at least three light emitting components. The at least two light emitting components each emit light within a range of about 400 nm to about 1100 nm, from about 450 nm to about 650 nm, and about 550 nm to about 950 nm. The at least one filter is a near-infrared cut-off filter. The at least two light emitting components are selected such that peak wavelengths of each of the at least two light emitting components are selected from a combination of wavelengths in which at least one has an absorption coefficient that is higher for oxyhemoglobin and at least one has an absorption coefficient that is higher for deoxyhemoglobin. The processor is configured to generate at least one of tissue oxygenation, photoplethysmograph waveform, heart rate, motion, total hemoglobin or hydration. In an example, the ambient light blocking component is a light blocking material having a first layer selected from Dartex®, neoprene, and combinations thereof and a second layer of an elastane, such as LYCRA®. The ambient light blocking component is configured to block light extending to at least 60 mm from the center of the photodiode.

In one example, the device can further include a body that includes a light blocking material and a protrusion surrounding the at least two light emitting components and the at least one photodiode to prevent external light from entering a region bounded by the protrusion. The protrusion and the body can be co-molded and made of the same light blocking material. In one example, the light blocking material can be a plastic containing carbon black.

The present disclosure further provides for a material configured to block ambient light for use with an optical device non-invasively perform tissue monitoring. In an example, the material includes a first layer selected from Dartex®, neoprene, and combinations thereof, and a second layer of an elastane, such as LYCRA®. The optical device with the light blocking material includes at least two light emitting components and at least one photodiode configured to receive reflected light generated from the at least two light emitting components. The material is configured to block light extending to at least 60 mm from the center of the photodiode.

The present disclosure generally relates to a non-invasive optical-electronic device. The device can be configured to determine a level of a biological indicator within tissue or blood vessels using near-infrared or visible light. Examples of non-invasive optical-electronic devices configured to determine levels of biological indicators are described in U.S. Pat. No. 8,996,088 entitled APPARATUS AND METHOD FOR IMPROVING TRAINING THRESHOLD, the entire contents of which are incorporated herein by reference. In order to optically monitor the tissue, ambient light from the surrounding environment must be blocked. The device disclosed herein is configured to block ambient light. The device itself can perform the ambient light blocking. Alternatively, the device can be worn with an ambient light-blocking sleeve as described in this disclosure.

Wearable athletic monitoring according to the present disclosure includes the use of non-invasive devices that are robust to motion and intense ambient light, especially exposure to sun light. Near infrared (NIR) light in the range from 650 nm to 1100 nm presents the combined advantage of penetrating deep into biological tissue while been effectively detected by low-cost “p-doped,” intrinsic, “n-doped” (PIN) Silicon photodiodes.

The optical-electronic device can be used by itself or in combination with other optical-electronic devices or biosensors. The optical-electronic device can be configured to determine physiological parameters of a user during exercise. It is to be understood, however, that the non-invasive optical-electronic device can also be used in other applications without departing from the principles of the present disclosure, including microcirculation analysis, newborn perfusion deficit, assessment of hemorrhage and shock, monitoring of fluid resuscitation, cognitive studies, cerebral oxygenation monitoring during cardiothoracic procedures, muscular oxygenation monitoring to diagnose acute and chronic compartment syndrome, and the monitoring of coronary artery disease (CAD) and other cardiovascular diseases.

The present disclosure generally relates to a non-invasive optical-electronic device configured to measure physiological parameters of a user. In one example, an optical-electronic device for determining the level of a biological indicator within tissues or blood vessels using Near Infrared Spectroscopy (NIRS) is provided. In another example, an optical-electronic device for determining the level of a biological indicator within tissues or blood vessels using visible light is provided. In both examples, the device includes a processor that calculates a relative match between a spectral data set representative of received light and a predetermined spectral data set of one or more chromophores. The optical-electronic device can be configured to transmit an alert to an output device. The optical-electronic device can be further configured to communicate a level of biological indicator to a user in real-time.

In at least one example, an optical-electronic device for determining the level of a biological indicator within tissues or blood vessels, is provided. The optical-electronic device is configured to alert a user to the existence of extraneous factors which interfere with the identification and/or determination of one or more biological indicators. The device can determine the existence of an extraneous factor by determining a modulus of a residual of the fit of a projection onto a matrix containing the spectra representative of a predetermined data set of one or more chromophores. The device can also determine the existence of an extraneous factor by determining the relative match of a spectral data set representative of received light and the null space for a matrix containing the spectra representative of a predetermined data set of one or more chromophores.

In a further example, an optical-electronic device can be configured to determine the level of one or more biological indicators during exercise and other physical conditions.

In at least one example, a method of determining the level of one or more biological indicators using an optical-electronic device can be configured to determine the level of a biological indicator within tissue or blood vessels using NIRS. The method of determining the level of one or more biological indicators using an optical-electronic device can be configured to determine the level of a biological indicator within tissue or blood vessels using visible light. Each of these methods includes the step of calculating a relative match between a spectral data set representative of received light and a predetermined spectral data set of one or more chromophores. The method can further include transmitting an alert to an output device. The method can further include communicating a level of a biological indicator to a user in real-time.

In another example, a device is configured to determine the level of a biological indicator within tissues or vessels is provided. The device includes at least two light emitting components, configured to emit light into a tissue. The device further includes a photodetector, configured to detect the light back-scattered from the tissue and transmitting data representative of the received reflected light. The device further includes a processing component configured to process the data representative of the received reflected light with a processor having a non-transitory storage medium configured to store instructions to cause the processor to receive the data representative of the received reflected light. The device can also include a processor configured to execute instructions to compare the data representative of the received reflected light to a predetermined spectral data set of one or more chromophores corresponding to a biological indicator. The device can also include a processor configured to execute instructions to calculate a relative match between the data representative of the received reflected light to the predetermined spectral data set. The device can also include a processor configured to execute instructions to estimate a biological indicator based on the calculated relative match. The device can also include a processor configured to execute instructions to transmit the level of the biological indicator to an output device. The device can also include a processor configured to execute instructions to transmit an alert to an output device.

In a further example, a method of calibrating optical data can be used to determine a level of a biological indicator. The method includes generating a calibration factor to convert detected light into optical densities for a given current, emitting light from at least two emitters into a tissue, where the two emitters are separated by a known distance. The method can also include converting detected light data into optical densities for a given current using the calibration factor. The method can include converting the optical densities into effective attenuation coefficients. The method can also include converting the effective attenuation coefficients into absorption coefficients using a reduced scattering coefficient obtained for the tissue being monitored, wherein the absorption coefficient corresponds to the attenuation properties of the tissue, and using the relative match of the absorption coefficient to predetermined spectral data to determine the level of a biological indicator in tissue. The method can further include communicating the level of a biological indicator to a user in real-time.

In another example, a method of determining a user-specific measure of a biological indicator in a tissue using a predetermined set of user-specific parameters is provided. The method includes generating a set of user-specific parameters based on that user's biological indicator data collected during an assessment using an optical-electronic device configured to capture optical data of a tissue. The method can also include storing the set of user-specific parameters on a server. The method can include measuring a biological indicator in the tissue of the user during a physical activity using an optical-electronic device configured to capture the optical data of a tissue. The method can also include calculating a user-specific measure of the biological indicator using the set of user-specific parameters stored on the server. The method can further include transmitting an alert to an output device, wherein the alert is configured to notify the user of a user-specific measure of biological indicator. The method can further include communicating a level of a biological indicator to a user in real-time.

In a further example, a device is configured to monitor tissue using near-infrared light is disclosed. The device includes at least two light emitting components. The light emitting components can be low-power lasers, light emitting diodes (LEDs) or quasi-monochromatic light sources. In one example, the light emitting components emit light in the near infrared spectrum. In another example, the light emitting components emit light at wavelengths in the visible spectrum. In one example, light is emitted by the light emitting components within the range of about 400 nm to about 1100 nm, from about 450 nm to about 650 nm, and about 550 nm to about 950 nm. These wavelengths are in a range over which tissue highly absorbs light, resulting in strong absorption of ambient light, thus requiring reduced light-blocking materials or fabrics. In one example, light is emitted by the light emitting components within the range of about 400 nm to about 650 nm. In one example, the at least two light emitting components emit light at wavelengths of 440 nm and 630 nm. The molar extinction coefficients of oxyhemoglobin and deoxyhemoglobin have higher absorption of light within the above mentioned range of wavelengths. In another example, the wavelengths are selected from a combination of wavelengths in which at least one has an absorption coefficient that is higher for oxyhemoglobin and at least one has an absorption coefficient that is higher for deoxyhemoglobin. In another example, the device includes at least three light-emitting components. In an example with at least three light-emitting components, the light-emitting components emit light at wavelengths of about 505 nm, about 470 nm and about 630 nm. In yet another example, the light-emitting components can emit light at wavelengths selected from about 550 nm, about 950 nm, and about 650 nm. Without being limited to a particular theory, a wavelength of about 550 nm can be used for a high hemoglobin absorption region but may also affected by melanin concentration, a wavelength of about 950 nm can be used for a high water absorption region but may have low hemoglobin absorption and be weakly affected by melanin, and a wavelength of about 650 nm can be used for a large separation between oxy- and deoxyhemoglobin, enabling the determination of blood oxygenation. Since blood plasma is mostly water, a wavelength of about 950 nm can also work well in detecting heart rate, especially if combined with a wavelength of about 530 nm using the techniques described in this disclosure.

The device further includes at least one photodiode, an analog-to-digital converter component configured to digitize detected light, a component configured to process digitized signals, a processing unit for processing digitized signals, an ambient light blocking component, and at least one cut-off filter over the at least one photodiode. In a specific example, the at least one photodiode is a Silicon photodiode, and the cut-off filter is a near infrared cut-off filter.

The device can be used for monitoring at least one of tissue oxygenation, the photoplethysmograph (PPG) waveform, heart rate, motion, total hemoglobin or hydration. In one example, the device itself is made of light blocking material, and the ambient light blocking is performed by the device itself. The device can include a protrusion that can surround the light emitting components and the photodiodes to prevent external light from entering the region bounded by the protrusion. In an example, the protrusion can be a plastic containing carbon black or another light blocking material. The protrusion and the body of the device can be co-molded and made of the same light blocking material. In a further example, the device can be configured to transmit an alert to an output device. The device can be further configured to communicate a level of biological indicator to a user in real-time. Further, the device can be configured to transmit the optical signals it detects wirelessly or through a wire to another device, to the cloud based system, or to a server wherein the signals can be processed and/or stored.

In a second example, the disclosure provides for a device operable to non-invasively perform tissue monitoring. The device can include the light emitters emitting light at wavelengths in the visible spectrum, and a photodetector with an infrared cut-off filter over it collects the light back-scattered from the tissue. The at least one photodiode is spaced apart from the at least two light emitting components. The device further includes an analog-to-digital converter component operable to generate a digitized detected light signal based on data received from the at least one photodiode, a processor operable to execute instructions to process the digitized signal, and at least one NIR-cut filter mounted over the at least one photodiode. The visible wavelengths can be chosen so that at least one of them is an isobestic wavelength. In at least one example, the wavelengths are 440 nm, 505 nm and 650 nm.

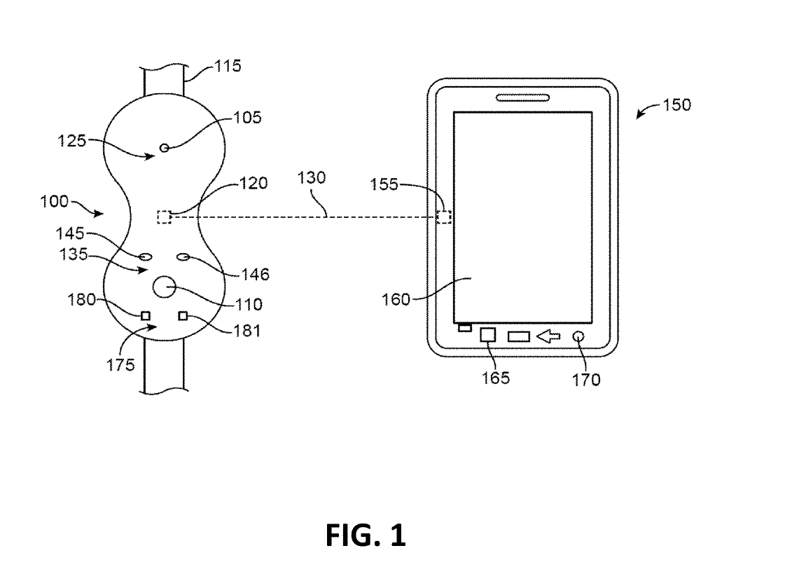

FIG. 1 illustrates a non-invasive optical-electronic device 100, according to an example of this disclosure. The device 100 can be attached to a portion, such as a muscle mass, of a user via a strap 115. The device 100 can be used with an optional output device 150, such as a smartphone (as shown), a smart watch, computer, mobile phone, tablet, a generic electronic processing and displaying unit, cloud storage, or a remote data repository via a cellular network or wireless Internet connection.

The device 100 includes a sensor 125 that is configured to determine the level of a biological indicator within tissue or blood vessels using NIRS. The sensor 125 includes an optical emitter 105 and an optical detector 110. In general, the sensor 125 uses two or more low-power lasers, light emitting diodes (LEDs) or quasi-monochromatic light sources and low-noise photodetecting electronics to determine the optical absorption of chromophores, such as water, hemoglobin in its multiple forms, including oxyhemoglobin (HbO2), deoxyhemoglobin (HHb), oxymyoglobin, deoxymyoglobin, cytochrome c, lipids, melanins, lactate, glucose, myoglobin (including myoglobin at least one of oxymyoglobin, deoxymyoglobin, and total myoglobin) or metabolites. The metabolites can include at least one of lactate and lactic acid. Cytochrome c can be used, for example, to track muscle adaptation to training. In another example, the sensor 125 can use a broad-spectrum optical source and a detector sensitive to the spectral components of light, such as a spectrometer, or a charge-coupled device (CCD) or other linear photodetector coupled with near-infrared optical filters.

The optical-electronic device 100 can be configured to include a second sensor 135 configured to measure photoplethysmography (PPT) of the user. The second sensor 135 includes an optical emitter 145 and an optical detector 146. The device 100 also includes a third sensor 175 configured to measure electrocardiography (EKG) and derived systolic time intervals (STI) of the user. The third sensor 175 includes a first electrode 180 and a second electrode 181. The sensors 125, 135, 175 in the device 100 can measure NIRS parameters, electrocardiography, photoplethysmography, and derived systolic time intervals (STI) of the user. The optical-electronic device 100 also includes a processor that is configured to analyze data generated by the sensors 125, 135, 175 to determine a cardiac response to exercise and the supply, arteriovenous difference, utilization of oxygen by the muscle tissue and hydration of the muscular tissue.

In at least one example, the processor is configured to determine biological indicators, including, but not limited to a relative percentage, a saturation level, an absolute concentration, a rate of change, an index relative to a training threshold, and a threshold. In other cases, the processor is configured to determine perfusion characteristics such as pulsatile rhythm, blood volume, vascular tone, muscle tone, and angiogenesis from total hemoglobin and water measurements.

The device 100 can include a power supply, such as a battery, to supply power to the sensors 125, 135, 175 and other components in the device 100. In one example, the sensor 125 has a skin contact area of 3.5″×2″. In other examples, the device 100 can be sized to be on the user's wrist so that there is a skin contact area of 1.5″33 1.5″. Additionally, other changes in dimensions are considered within the scope of this disclosure.

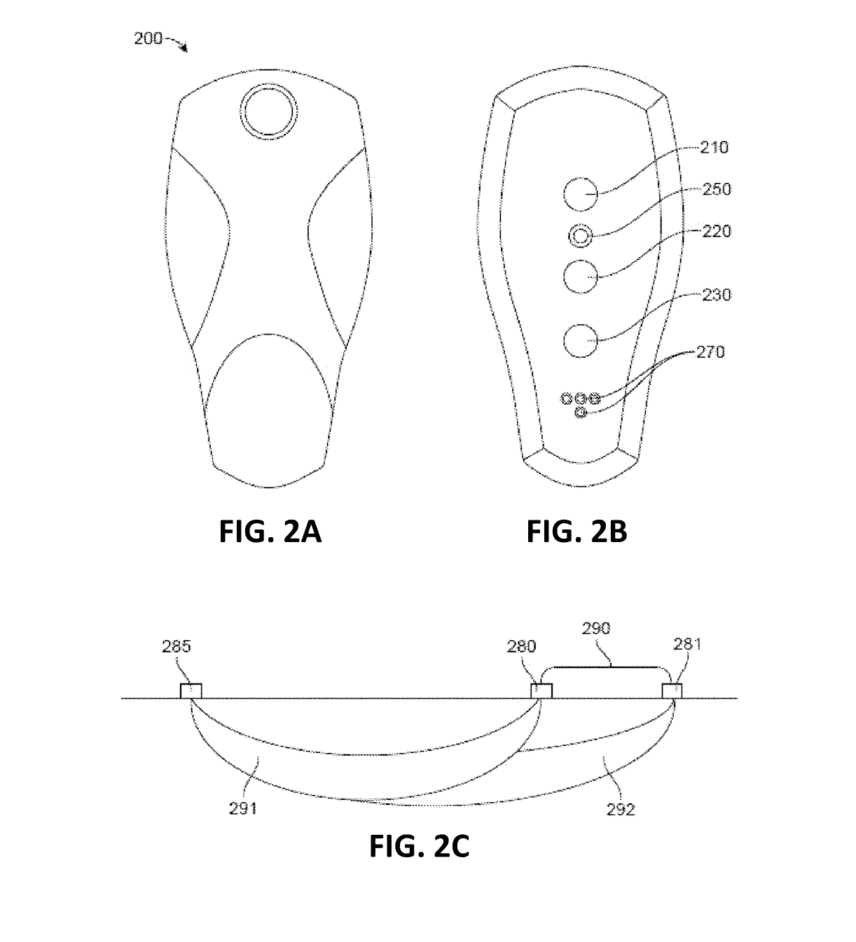

FIG. 2 illustrates a non-invasive optical-electronic device 200, according to an alternative example of this disclosure. The device 200 is configured to be worn on a limb of a user, such as on the calf muscle of a user's leg or the bicep of a user's arm. In at least one example, the device 200 can be optimized to a given limb for increased accuracy. In other examples, the device 200 can be optimized based on the size, gender, or age of the user. In still other examples, a variety of the above optimizations can be implemented for a given device. FIG. 2A illustrates the front of the optical-electronic device. FIG. 2B illustrates the back of the optical-electronic device, including emitters 220, 230, 250 and photodetector 210. The device 200 also includes data and charging contacts 270. In at least one example, the data and charging contacts 270 can be used to electrically detect if the sensor is making contact with the skin of a user. The presence of multiple emitters 220, 230, 250 on the optical-electronic device allows for spatially-resolved data gathering in real-time. The optical-electronic device 200 can be configured to determine the optical absorption of chromophores, such as water, hemoglobin in its multiple forms, including oxyhemoglobin (HbO2), deoxyhemoglobin (HHb), oxymyoglobin, deoxymyoglobin, cytochrome c, lipids, melanins, lactate, glucose, or metabolites.

FIG. 2C illustrates a spatially-resolved NIRS sensor that can be included on the non-invasive optical-electronic device 200, according to an example of the disclosure. As shown in FIG. 2C, the spatially-resolved NIRS sensor includes light emitters 280 and 281 which emit light that is scattered and partially absorbed by the tissue. Each emitter 280, 281 can be configured to emit a single wavelength of light or a single range of wavelengths. In at least one example, each emitter 280, 281 can be configured to emit at least three wavelengths of light or at least three ranges of wavelengths. Each emitter 280, 281 can include one or more light emitting diodes (LEDs). Each emitter 280, 281 can include a low-powered laser, LED, or a quasi-monochromatic light source, or any combination thereof. Each emitter 280, 281 can also include a light filter.

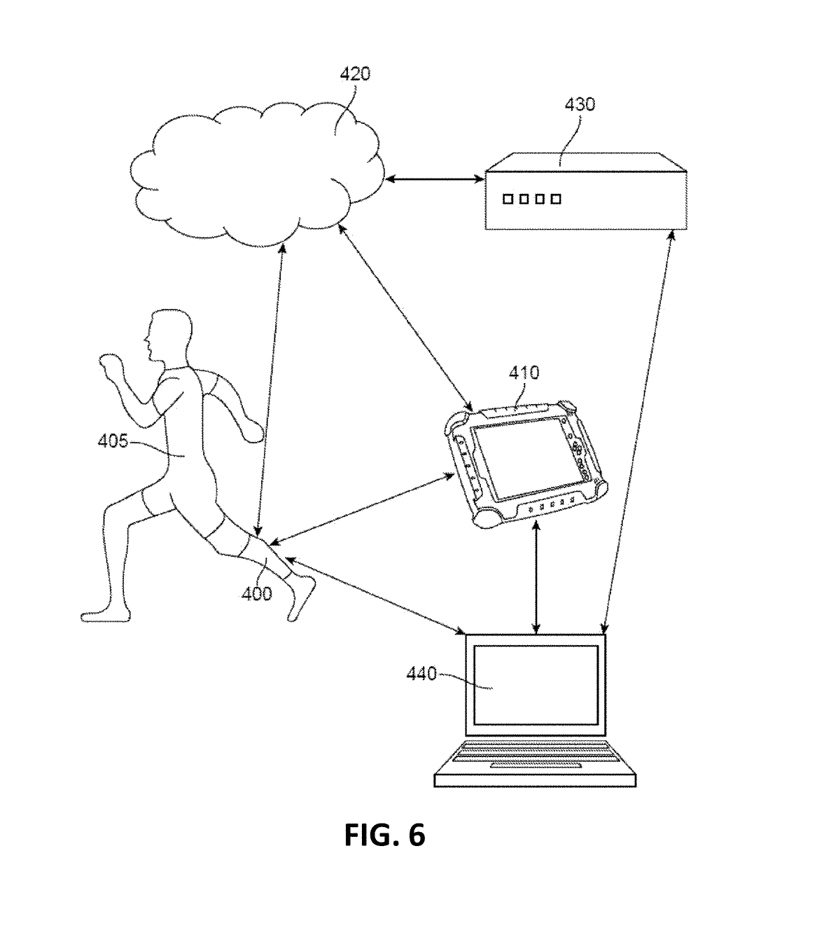

A fraction of the light emitted by emitters 280 and 281 is detected by photodetector 285, as illustrated by the parabolic or “banana shaped” light arcs 291 and 292. Emitters 280, 281, are separated by a known distance 290 and produce a signal that is later detected at photodetector 285. The detected signal is used to estimate the effective attenuation and absorption coefficients of the underlying tissue as described later in FIG. 6, for example at blocks 640 and 650. In at least one example, the known distance 290 is about 12 mm. In another example, multiple distances are used and at least one of them is about 15 mm and at least another one of them is about 27 mm. In other examples, the known distance can be selected based on a variety of factors, which can include the wavelength of the light, the tissue involved, or the age of the user.

The optical-electronic device 200 disclosed herein can have different numbers of emitters and photodetectors without departing from the principles of the present disclosure. Further, the emitters and photodetectors can be interchanged without departing from the principles of the present disclosure. Additionally, the wavelengths produced by the LEDs can be the same for each emitter or can be different.

In at least one example, the device 200 is used for the monitoring of physiological parameters of a user during a physical activity. Use of the device 200 is particularly relevant in endurance type sports, such as running, cycling, multisport competition, rowing, but can also be used in other physical activities. The device 200 can be configured to wirelessly measure real-time muscle parameters during physical exercise. The device 200 can be secured to a selected muscle group of the user, such as the leg muscles of the vastus lateralis or gastrocnemius, which are primary muscle groups of running and cycling.

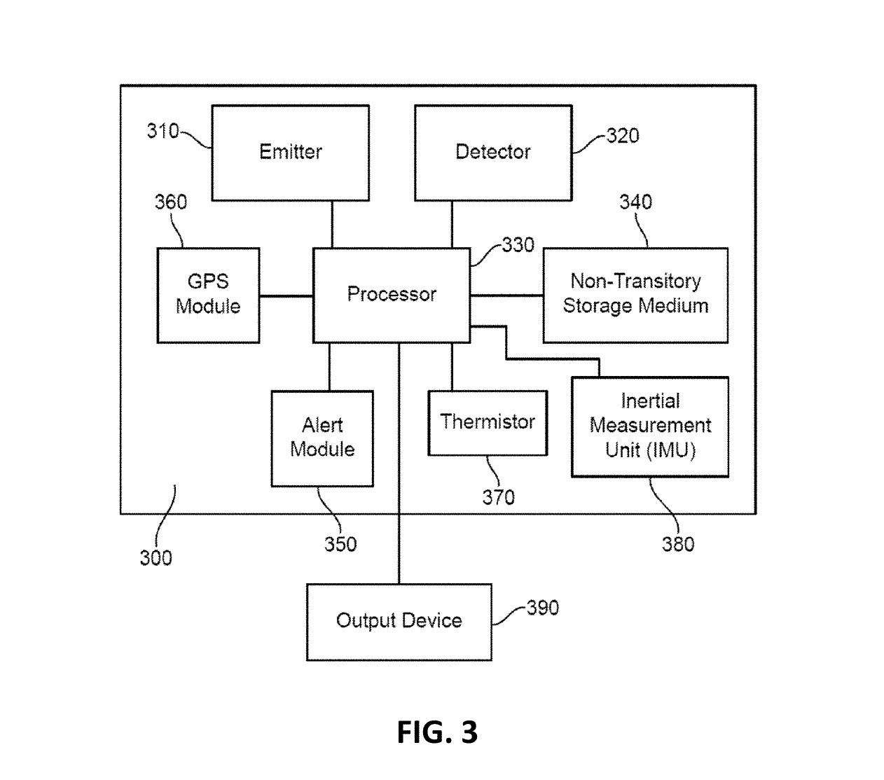

FIG. 3 illustrates the components of an optical-electronic device 300 according to an example of this disclosure. As shown in FIG. 3, the optical-electronic device includes an emitter 310 and detector 320, which are coupled to a processor 330. The processor 330 is coupled to a non-transitory storage medium 340. The device 300 is coupled to an output device 390.

The emitter 310 delivers light to the tissue and the detector 320 collects the optically attenuated signal that is back-scattered from the tissue. In at least one example, the emitter 310 can be configured to emit at least three separate wavelengths of light. In another example, the emitter 310 can be configured to emit at least three separate bands or ranges of wavelengths. In at least one example, the emitter 310 can include one or more light emitting diodes (LEDs). The emitter 310 can also include a light filter. The emitter 310 can include a low-powered laser, LED, or a quasi-monochromatic light source, or any combination thereof. The emitter can emit light ranging from infrared to ultraviolet light. As indicated above, the present disclosure uses NIRS as a primary example and the other types of light can be implemented in other examples and the description as it relates to NIRS does not limit the present disclosure in any way to prevent the use of the other wavelengths of light.

The data generated by the detector 320 can be processed by the processor 330, such as a computer processor, according to instructions stored in the non-transitory storage medium 340 coupled to the processor. The processed data can be communicated to the output device 390 for storage or display to a user. The displayed processed data can be manipulated by the user using control buttons or touch screen controls on the output device 390.

The optical-electronic device 300 can include an alert module 350 configured to generate an alert. The processor 330 can send the alert to the output device 390 or the alert module 350 can send the alert directly to the output device 390. In at least one example, the optical-electronic device 300 can be configured so that the processor 330 is configured to send an alert to the output device 390 without the device including an alert module 350.

The alert can provide notice to a user, via a speaker or display on the output device 390, of a change in biological indicator conditions or other parameter being monitored by the device 300, or the alert can be used to provide an updated biological indicator level to a user. In at least one example, the alert can be manifested as an auditory signal, a visual signal, a vibratory signal, or combinations thereof. In at least one example, an alert can be sent by the processor 330 when a predetermined biological indicator event occurs during a physical activity.

In at least one example, the optical-electronic device 300 can include a Global Positioning System (GPS) module 360 is configured to determine a geographic position and tag the biological indicator data with location-specific information. The optical-electronic device 300 can also include a thermistor 370 and an inertial measurement unit (IMU) 380. The IMU 380 can be used to measure, for example, gait performance of a runner or pedal kinematics of a cyclist, as well as physiological parameters of a user during a physical activity. The thermistor 370 and IMU 380 can also serve as independent sensors configured to independently measure parameters of physiological threshold. The thermistor 370 and IMU 380 can also be used in further algorithms to process or filter the optical signal.

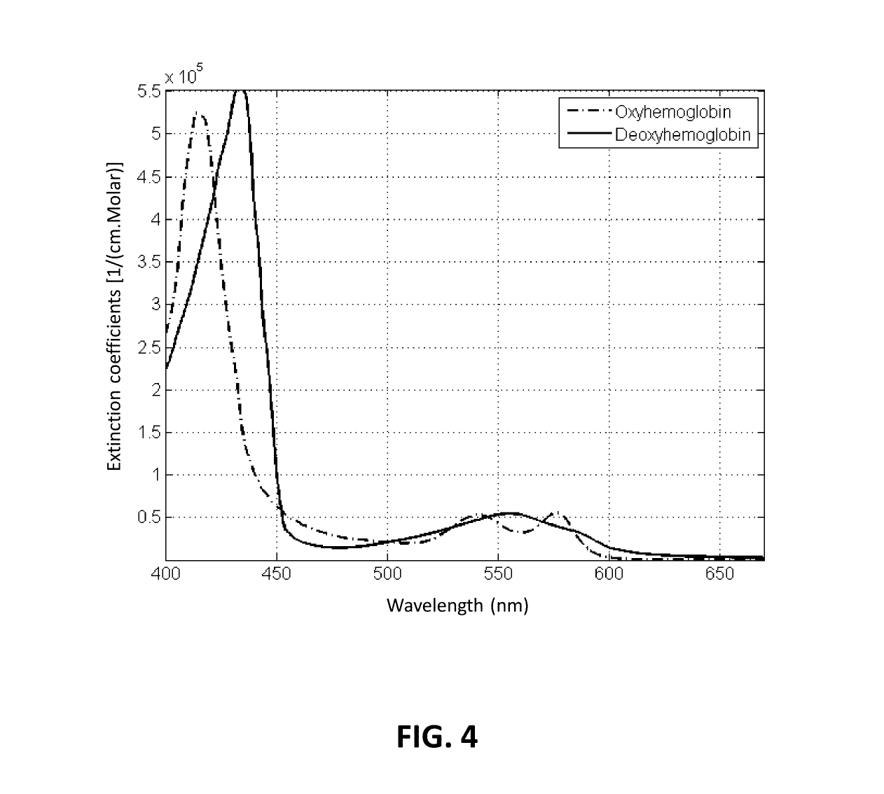

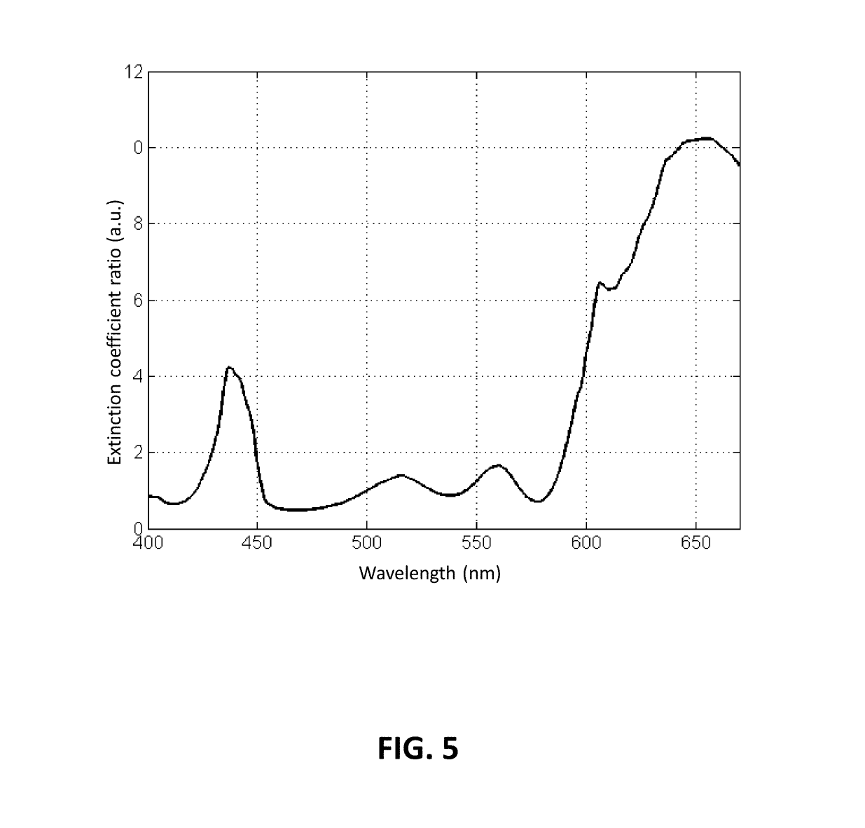

The device disclosed herein can have different numbers of emitters and photodetectors without departing from the principles of the present disclosure. Further, the emitters and photodetectors can be interchanged without departing from the principles of the present disclosure. Additionally, the wavelengths produced by the LEDs can be the same for each emitter or can be different. In one example, the wavelengths selected are 505 nm, 440 nm and 630 nm. These wavelengths are selected based on the plots in FIGS. 4 and 5.

FIG. 5 shows the ratios of the molar extinction coefficients of oxyhemoglobin and deoxyhemoglobin. Points with high ratio correspond to points where one can more easily distinguish between the two heme species and, hence, more easily determine the oxygenation of blood. Points wherein the ratio equals unity are known as isobestic points which, being insensitive to the state of oxygenation of blood, are useful in determining the total hemoglobin concentration. When selecting wavelengths that lie within the range over which light (including ambient) is effectively absorbed, while presenting the minimum number of wavelengths with the requirements that at least one of them is isobestic, at least one has the smallest possible ratio of molar extinction coefficients, at least one has the largest possible ratio of molar extinction coefficients and restricting those wavelengths to those that are commercially available, the set of wavelengths 505 vnm, 440 nm and 630 nm to fulfill all selection criteria. Furthermore, wavelengths shorter than 440 nm are generally not with the scope of the present disclosure because of the extremely high tissue absorption within the range from 440 nm to 400 nm, as shown in FIG. 4. The light sources in the ultraviolet (UV) region of the spectrum can cause photochemical harm to biological tissue.

FIG. 5 shows a plot of the ratio of the deoxyhemoglobin and oxyhemoglobin extinction coefficients as a function of wavelength over the visible range of wavelengths. The isobestic points are represented by the wavelengths in which the plot crosses unity—that is, deoxyhemoglobin and oxyhemoglobin present the same extinction coefficients. Of those points the one at 500 nm represents the isobestic point at which the local slope is smallest and, hence, the ratio is least sensitive to small deviations in wavelength. At least two points are needed in order to measure oxygen saturation. Hence, the peaks at 438 nm and 650 nm are selected not only because they represent the largest peaks available within this wavelength ratio but also because the peaks at 438 nm and 650 nm represent points wherein deoxyhemoglobin and oxyhemoglobin have inverted extinction coefficients (see FIG. 4). This way it is possible to monitor oxygen saturation using only visible light. Furthermore, NIR-cut-off optical filters provide ambient light blocking without requiring the extensive use of additional light blocking materials.

The data generated by the detector can be processed by the processing unit, such as a computer processor, according to instructions stored in a non-transitory storage medium coupled to the processing unit. The processed data can be communicated to the output device for storage or display to a user. The displayed processed data can be manipulated by the user using control buttons or touch screen controls on the output device.

The alert can provide notice to a user, via a speaker or display on the output device, of a change in biological indicator conditions or other parameter being monitored by the device, or the alert can be used to provide an updated biological indicator level to a user. In at least one example, the alert can be manifested as an auditory signal, a visual signal, a vibratory signal, or combinations thereof. In at least one example, an alert can be sent by the processing unit when a predetermined biological indicator event occurs during a physical activity.

In at least one example, the device can include a Global Positioning System (GPS) module configured to determine a geographic position and tag the biological indicator data with location-specific information. The device can also include a thermistor and an IMU. The IMU can be used to measure, for example, gait performance of a runner or pedal kinematics of a cyclist, as well as physiological parameters of a user during a physical activity. The thermistor and IMU can also serve as independent sensors configured to independently measure parameters of physiological threshold. The thermistor and IMU can also be used in further algorithms to process or filter the optical signal.

FIG. 6 illustrates an environment within which the noninvasive optical-electronic device 400 can be implemented, according to an example of this disclosure. As shown in FIG. 6, the optical-electronic device 400 is worn by a user to determine biological indicator levels during a physical activity. The optical-electronic device 400 is depicted as being worn on the calf of a user 405, however, the optical-electronic device 400 can be worn on any portion of the user suitable for monitoring biological indicator levels. The device 400 can be used with an output device 410, such as a smartphone (as shown), a smart watch, computer, mobile phone, tablet, a generic electronic processing and displaying unit, cloud storage, or a remote data repository via a cellular network or wireless Internet connection.

As shown in FIG. 6, the optical-electronic device 400 communicates with a output device 410 so that data collected by the optical-electronic device 400 is displayed or transferred to the output device 410 for communication of real-time biological indicator data to the user 405. In at least one example, an alert can be communicated from the device 400 to the output device 410 so that the user 405 can be notified of a biological indicator event. Communication between the device 400 and the output device 410 can be via a wireless technology, such as BLUETOOTH®, infrared technology, or radio technology, or can be through a wire. Transfer of data between the optical-electronic device 400 and the output device 410 can also be via removable storage media, such as a secure digital (SD) card. In at least one example, a generic display unit can be substituted for the output device 410.

The optical-electronic device 400 also communicates with a personal computing device 440 or other device configured to store or display user-specific biological indicator data. The personal computing device 440 can include a desktop computer, laptop computer, tablet, smartphone, smart watch, or other similar device. Communication between the device 400 and the personal computing device 440 can be via a wireless technology, such as BLUETOOTH®, infrared technology, or radio technology. In other examples, the communication between the device 400 and the personal computing device 440 can be through a wire or other physical connection. Transfer of data between the optical-electronic device 400 and the personal computing device 440 can also be via removable storage media, such as an SD card.

The output device 410 can communicate with a server 430 via a network 420, allowing transfer of user-specific biological indicator data to the server 430. The output device 410 can also communicate user-specific biological indicator data to cloud-based computer services or cloud-based data clusters via the network 420. The output device 410 can also synchronize user-specific biological indicator data with a personal computing device 440 or other device configured to store or display user-specific biological indicator data. The output device 410 can also synchronize user-specific biological indicator data with a personal computing device 440 or other device configured to store and display user-specific biological indicator data. Alternatively, the personal computing device 440 can receive data from a server 430 or cloud-based computing service via the network 420.

The personal computing device 440 can communicate with a server 430 via a network 420, allowing the transfer of user-specific biological indicator data to the server 430. The personal computing device 440 can also communicate user-specific biological indicator data to cloud-based computer services or cloud-based data clusters via the network 420. The personal computing device 440 can also synchronize user-specific biological indicator data with the output device 410 or other device configured to store or display user-specific biological indicator data.

The optical-electronic device 400 can also directly communicate data via the network 420 to a server 430 or cloud-based computing and data storage service. In at least one example, the device 400 can include a GPS module configured to communicate with GPS satellites (not shown) to obtain geographic position information.

The optical-electronic device 400 can be used by itself or in combination with other optical-electronic devices or biosensors. For example, the optical-electronic device 400 can be used in combination with heart rate (HR) biosensor devices, foot pod biosensor devices, and/or power meter biosensor devices. The optical-electronic device 400 can also be used in combination with ANT+™ wireless technology and devices that use ANT+™ wireless technology. The optical-electronic device 400 can be used to aggregate data collected by other biosensors including data collected by devices that use ANT+™ technologies. Aggregation of the biosensor data can be via a wireless technology, such as BLUETOOTH®, infrared technology, or radio technology, or can be through a wire.

The biosensor data aggregated by the optical-electronic device 400 can be communicated via a network 420 to a server 430 or to cloud-based computer services or cloud-based data clusters. The aggregated biosensor data can also be communicated from the optical-electronic device 400 to the output device 410 or personal computing device 440.

In at least one example, the optical-electronic device 400 can employ machine learning algorithms by comparing data collected in real-time with data for the same user previously stored on a server 430, output device 410, or in a cloud-based storage service. The machine learning algorithm can also be performed on or by any one of the output device 410, cloud-based computer service, server 430, or personal computing device 440, or any combination thereof.

According to this disclosure, determination of the level of a biological indicator within tissue or blood vessels is achieved by calculating a relative match, or indices, between the spectral data received at the detector with a predetermined spectral data set of one or more chromophores corresponding to the biological indicator. In at least one example, the predetermined spectral data set corresponds to the signal spectra of specific analytes that can be readily obtained from the literature. See for example, Analyt. Biochem. Vol 227, pp. 54-68 (1995). The relative match calculation is performed by calculating a projection of the spectral data set captured from a user in the direction of the predetermined spectral data set in order to calculate an index that reflects the proximity of the match. The spectral projection method can be used to calculate a relative percentage level of a biological indicator or, with proper calibration, can be used to calculate the absolute concentration of a biological indicator.

The spectral projection method of determining the level of a biological indicator can be implemented mathematically using the inner product method which will be explained, by way of example, using the Total Oxygenation Index (TOI) as the biological indicator of interest. TOI is the ratio of the oxygenated hemoglobin (HbO2) to total hemoglobin (tHb), where total hemoglobin (tHb) is equal to the combined concentrations of the oxygenated hemoglobin (HbO2) and the chromophore deoxygenated hemoglobin (HHb):

TOI=[HbO2]/[tHb] or TOI %=100*([HbO2]/[tHb]), where [tHb]=[HbO2]+[HHb].

TOI, as used herein, includes the more specific parameter, SmO2, which is the muscle oxygen saturation. SmO2 can also be the tissue oxygen saturation determined from optical measurements of muscle tissue. Both oxygenated hemoglobin (HbO2) and deoxygenated hemoglobin (HHb) are chromophores for which a spectral data set can be predetermined. The notation O(D) can be used to denote the predetermined spectral data for oxyhemoglobin (deoxyhemoglobin) at the same wavelengths for which the spectral data set for a user was measured at the detector, and U can be used to denote the measured data set, including an effective attenuation (μ_{eff}) or an effective absorption coefficient (μₐ). The inner product method of calculating the spectral projection can be calculated according to different mathematical methods, including, but not limited to, a direction cosine method, vector projection method, and a pseudo-inverse projection method:

### Direction Cosine Method

$$TOI = \frac{〈 U , O 〉}{〈 U , O + D \sqrt{\frac{〈 O , O 〉}{〈 D , D 〉}} 〉} ,$$

### Vector Projection Method

$$TOI = \frac{〈 U , O 〉}{〈 U , O + D \sqrt{\frac{〈 O , O 〉}{〈 D , D 〉}} 〉} ,$$

### Pseudo-Inverse Projection Method

$$TOI = \frac{〈 U , O - \frac{〈 O , D 〉}{〈 D , D 〉} D 〉}{〈 U , O [ 1 - \frac{〈 O , D 〉}{〈 D , D 〉} ] + D [ \frac{〈 O , O 〉}{〈 D , D 〉} - \frac{〈 O , D 〉}{〈 D , D 〉} ] 〉} .$$

All of these methods can be rewritten as

$$TOI = \frac{〈 U , O - aD 〉}{〈 U , O ( 1 - a ) + D ( b - a ) 〉}$$

where a and b are scalars defined as

$$i ) a = 0 , b = \sqrt{\frac{〈 O , O 〉}{〈 D , D 〉}} ; ii ) a = 0 , b = \frac{〈 O , O 〉}{〈 D , D 〉} ; and iii ) a = \frac{〈 O , D 〉}{〈 D , D 〉} and b = \frac{〈 O , O 〉}{〈 D , D 〉}$$

for the cosine, vector projection and pseudo-inverse methods, respectively.

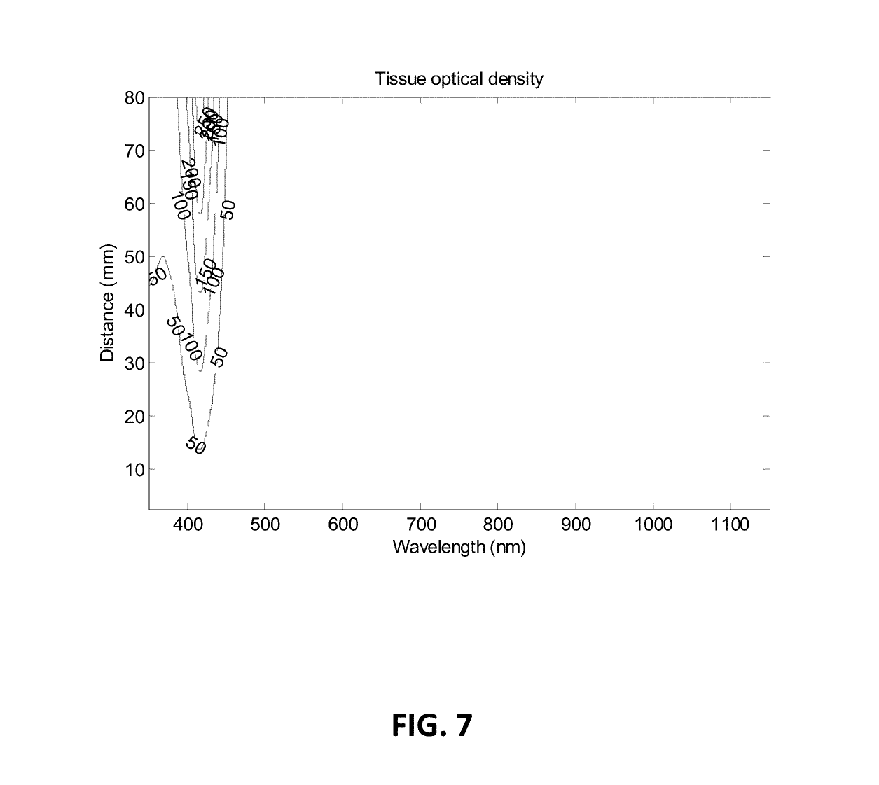

Light from the environment can be detected by the photodetector as well as the light emitted by the light emitters and back-scattered from the tissue. FIG. 7 is a plot showing the light absorbance by the wrist tissue of a subject across various light wavelengths. The low-absorbing tissue of the wrist does a good job of absorbing ambient visible light, but an especially poor job of absorbing ambient near-infrared light. In one example the filter is comprised of a NIR-cut-off filter and the LEDs are comprised of wavelengths within the range of visible light. This example presents the advantage that visible light is easily absorbed by tissue, limiting the need for additional light-blocking materials to stop ambient light from interfering with the device. As illustrated by FIG. 7, the tissue of the wrist is good at blocking many wavelengths of light itself. FIG. 7 also illustrates that a filter that blocks the ambient light in the infrared and near-infrared spectrum would protect the signal from interference from ambient light.

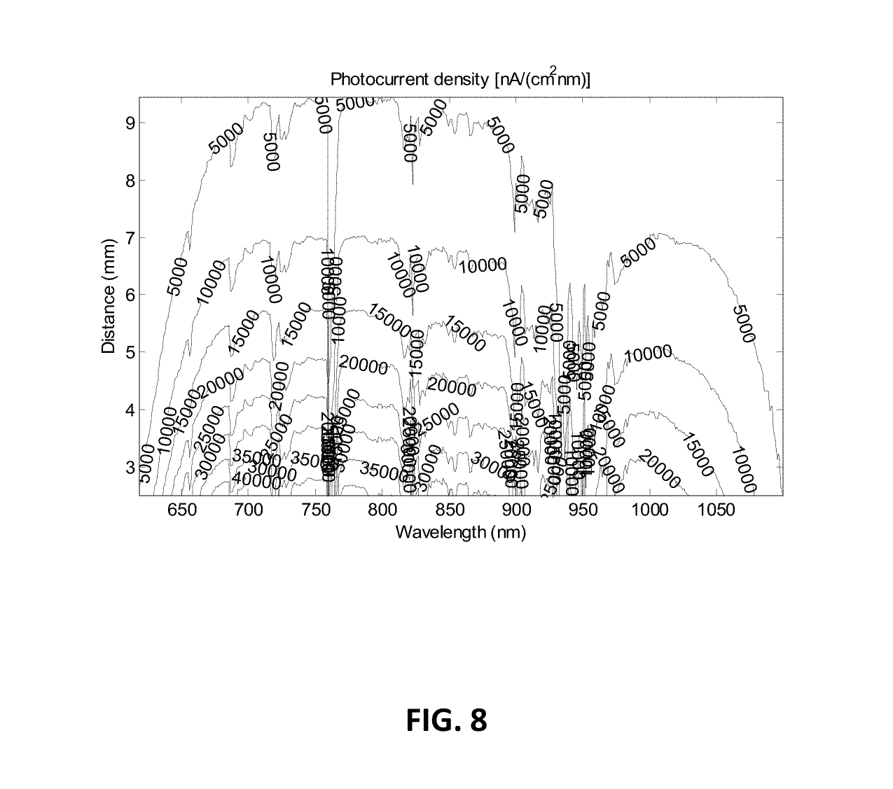

FIG. 8 illustrates the photocurrent generated in the device by ambient light in the near-infrared region. The ambient sunlight generates a large amount of photocurrent in the near-infrared region. This photocurrent interferes with the photocurrent generated by the light emitted by the light emitters and back-scattered from the tissue, and therefore ambient light needs to be blocked in order to generate an accurate signal.

The photocurrent generated in the device by ambient (sun) light can be represented by the following equation:

$${i}_{sun} = \frac{7 πA}{4} \int {\int }_{ρ = 17 , λ = 300}^{ρ = 80 , λ = 1120} {I}_{sun} ( λ ) T ( λ ) {R}_{r} ( λ , ρ ) P ( λ ) ℛ ( λ ) ρ d ρ d λ$$

where I is source irradiance, [symbol] is photodetector responsivity, Rᵣ is tissue transmission (T J Farrell, M S Patterson and B Wilson, ‘A Diffusion Theory Model of Spatially Resolved, Steady-State Diffuse Reflectance for the Noninvasive Determination of Tissue Optical Properties Invivo’, Medical physics, 1992, 879-88 <http://scitation.aip.org/content/aapm/journal/medphys/19/4/10.1118/1.596777> [accessed 30 Nov. 2014].), T is fabric transmission, P is filter transmission, ρ is distance, and A is the photodiode area. In one example, the signal photocurrent in the device can be represented by the following equation:

i_{LED j}^{m}=A∫I_{LED j}(λⱼ)P(λ) [symbol] (λⱼ)Rᵣ(λⱼ,ρₘ)dλ

where I is source irradiance, [symbol] is photodetector responsivity, Rᵣ is tissue transmission, T is fabric transmission, P is plastic transmission, ρ is distance, A is the photodiode area, m is the distance index (in this case {15,27}), j is the LED wavelength index [1,4]. For no interference in the signal photocurrent, minⱼₘ{i_{LED j}^{m}} must be >>i_{sun·}, which is achieved in this invention by reducing the amount of sunlight incident on the photodetector, as described next.

In another example, the signal photocurrent in the device can be represented by the following equation:

$$\begin{matrix}{i}_{LED j}^{m} = A \int {I}_{LED j} ( {λ}_{j} ) P ( λ ) ℛ ( {λ}_{j} ) {R}_{r} ( {λ}_{j} , {ρ}_{m} ) d λ \\ ≅ {R}_{r} ( {λ}_{j} , {ρ}_{m} ) \int {I}_{LED j} ( {λ}_{j} ) P ( λ ) ℛ ( {λ}_{j} ) d λ ≅ {R}_{r} ( {λ}_{cent j} , {ρ}_{m} ) {i}_{tot j}\end{matrix}$$

where I is source irradiance, [symbol] is photodetector responsivity, Rᵣ is tissue transmission, T is fabric transmission, P is plastic transmission, ρ is distance, A is the photodiode area, m is the distance index (in this case {15,27}), j is the LED wavelength index [1,4]. For no interference in the signal photocurrent, minⱼₘ{i_{LED j}^{m}} must be >>i_{sun·}, which is achieved in this disclosure by reducing the amount of sunlight incident on the photodetector, as described next.

The disclosed device is configured to block ambient light in two ways. In a first example, the device is made of plastic containing carbon black, which readily absorbs light in the visible and near infrared spectrum. The device can also include a protrusion surrounding the at least two light emitting components and the at least one photodiode to prevent external light from entering a region bounded by the protrusion. The body of the device and the protrusion can be co-molded from the same material, such as the plastic containing carbon black or a light blocking fabric.

Additionally, the device can be worn in conjunction with a sleeve made of a material or fabric composition that blocks ambient light. The sleeve can be made using a variety of different materials that are configured to provide the appropriate light blocking that is desired for the application. Additionally, the type of material can be chosen to provide appropriate water resistance, waterproofing, flexibility, and/or elasticity. The type of material, the combination of materials, and the color of material can be chosen to provide appropriate ambient light blocking. The light blocking material can include a first layer and a second layer. In one example, a polyurethane coating can be applied to a knitted fabric and form a layer of the material. In another example, at least one layer of the light blocking material can be a flexible, high optical density material. In some examples, the flexible, high optical density material can be DARTEX®, neoprene, or combinations thereof. In an example, if the light blocking material includes neoprene then it may not include the polyurethane coating. The second layer can be added to the first layer and can be in the form of an elastane or spandex, such as LYCRA®. In an example, the light blocking material can be made of a first layer of DARTEX® or neoprene and a second layer of elastane. In one example, the sleeve can be made of DARTEX® and LYCRA®, and in another example, the sleeve can be made of neoprene and LYCRA®. The color of at least one of the layers of the material in the sleeve can be black, gray, or another dark color to assist in blocking ambient light. In an example, the combination of the first layer and second layer is configured to block light extending to at least 60 mm from the center of the photodiode.

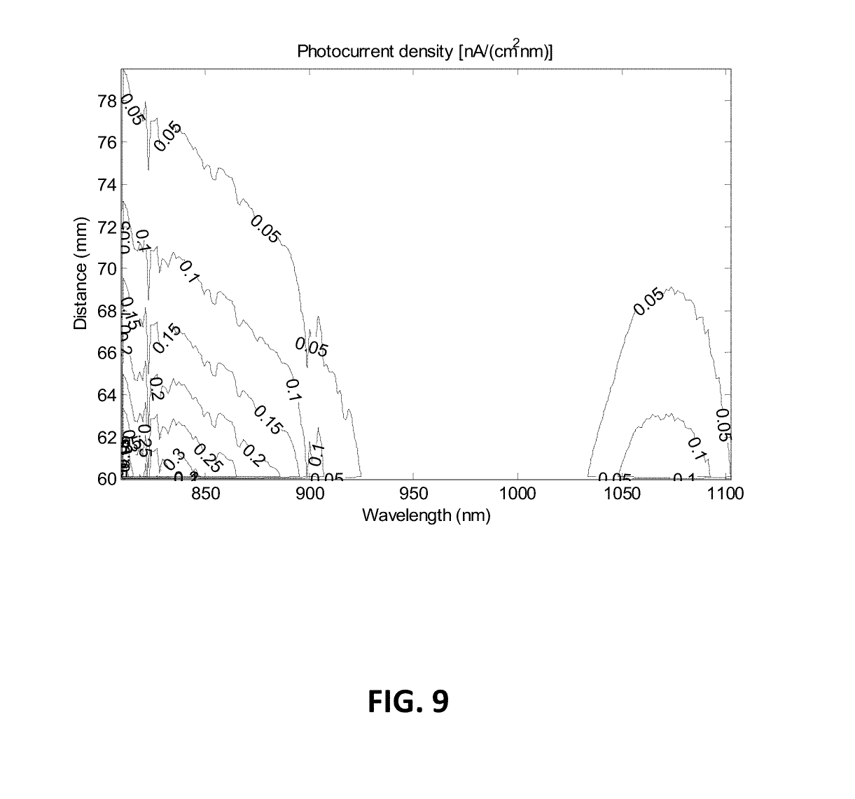

FIG. 9 is a plot of the photocurrent generated in the device when the device is enclosed within a radius of 60 mm of the fabric composition of this disclosure. As shown in FIG. 9, the photocurrent generated by ambient light is greatly reduced when the device is used in combination with the fabric sleeve.

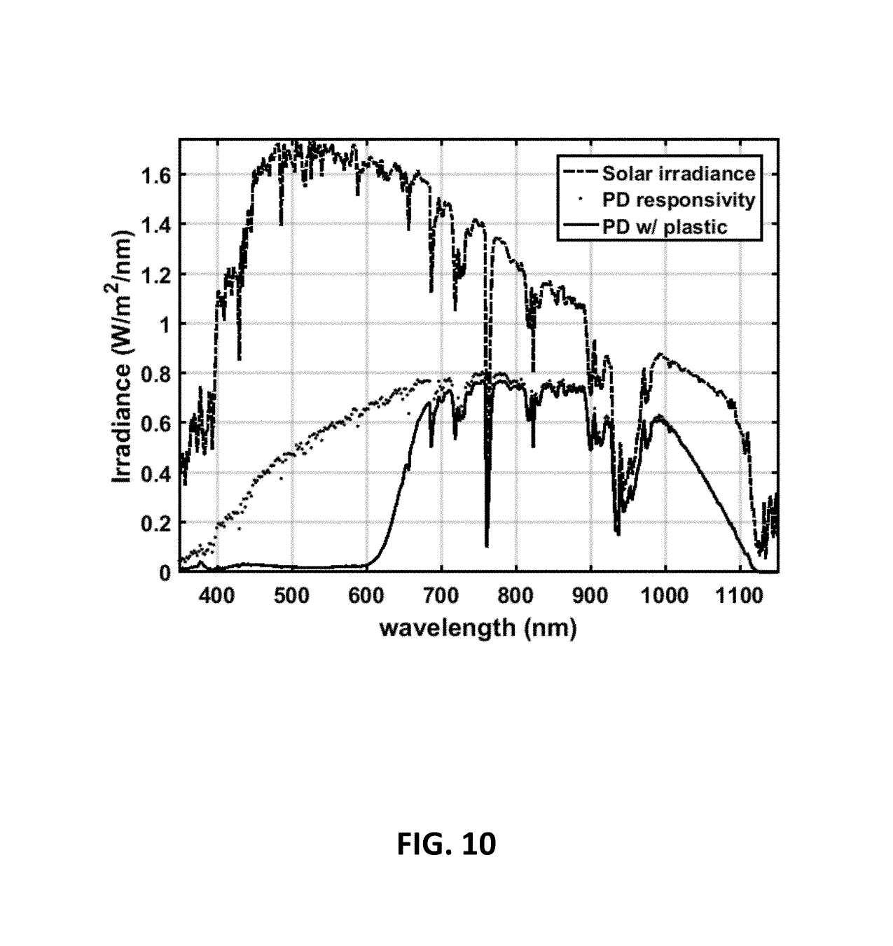

FIG. 10 shows a plot of the effect of ambient sunlight on a photodetector, and a photodetector with a plastic window. As shown in the plot, the plastic window effectively blocks ambient light in the range 300-600 nm wavelengths.

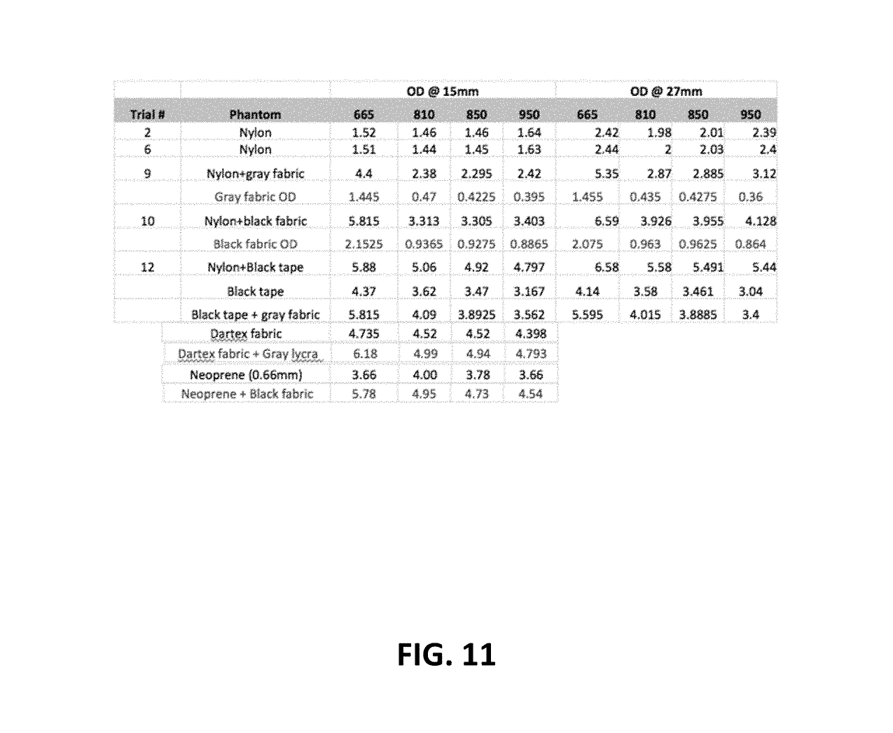

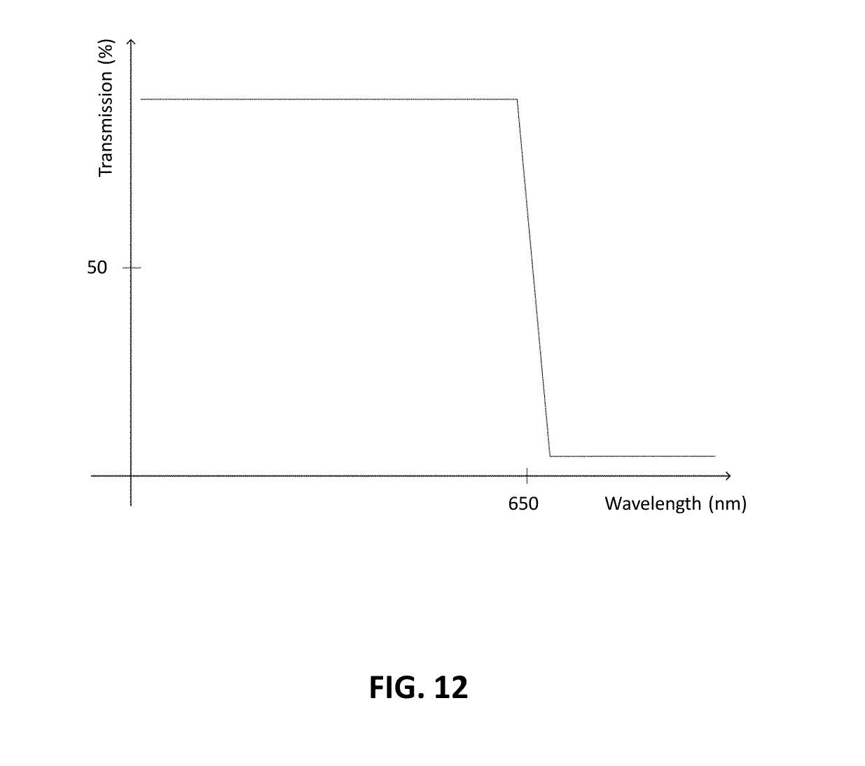

FIG. 11 is a table demonstrating the absorption properties of various types of fabrics. As one can see from the table, the gray LYCRA® absorbs about 2.5× of near-infrared light, while the black LYCRA® absorbs about 8× of near-infrared light. This can be compared to the gray LYCRA® absorbing about 25× of red light, and the black LYCRA® absorbing 125× of red light. Other materials used for ambient light blocking include Dartex®, which is preferred, and neoprene. Dartex® has an optical density of approximately 4.7 in the red and approximately 4.4 at 950 nm, while a piece of neoprene 0.66 m thick has an optical density of approximately 3.66 in the red and at 950 nm. Therefore, using LYCRA® alone the compression sleeve would be almost transparent to near-infrared light, highlighting the importance of adding layers of materials that effectively absorb infrared light. The sleeve of light blocking material can be attached to a large muscle. The sleeve fixes the wearable device firmly against large muscle, and can help decrease motion artifact in the optical signal.

What is claimed is:

1. A device configured to non-invasively perform tissue monitoring, the device comprising:
at least two light emitting components;
at least one photodiode configured to receive reflected light generated from the at least two light emitting components, wherein the at least one photodiode is spaced apart from the at least two light emitting components;
an analog-to-digital converter component configured to generate a digitized detected light signal based on data received from the at least one photodiode;
a processor configured to execute instructions to process the digitized signals;
an ambient light blocking component configured to surround the at least two light emitting components and the at least one photodiode to prevent external light from entering a region bounded by the ambient light blocking component; and
at least one filter mounted over the at least one photodiode.

2. The device of claim 1 comprising at least three light emitting components.

3. The device of claim 1 wherein the at least two light emitting components each emit light within a range of about 400 nm to about 1100 nm.

4. The device of claim 1 wherein the at least one filter is a near-infrared cut-off filter.

5. The device of claim 1 wherein the at least two light emitting components are selected such that peak wavelengths of each of the at least two light emitting components are selected from a combination of wavelengths in which at least one has an absorption coefficient that is higher for oxyhemoglobin and at least one has an absorption coefficient that is higher for deoxyhemoglobin.

6. The device of claim 1 wherein the processor is configured to generate at least one of tissue oxygenation, photoplethysmograph waveform, heart rate, motion, total hemoglobin or hydration.

7. The device of claim 1 wherein the ambient light blocking component comprises a light blocking material comprising:
a first layer of a flexible, high optical density material; and
a second layer of an elastane.

8. The device of claim 7, wherein the first layer is selected from Dartex®, neoprene, and combinations thereof.

9. The device of claim 7 wherein the elastane is LYCRA®.

10. The device of claim 1 wherein the ambient light blocking component is configured to block light extending to at least 60 mm from the center of the photodiode.

11. A device configured to non-invasively perform tissue monitoring, the device comprising:
a body that comprises a light blocking material;
at least two light emitting components mounted at least partially within the body;
at least one photodiode configured to receive reflected light generated from the at least two light emitting components, wherein the at least one photodiode is spaced apart from the at least two light emitting components;
an analog-to-digital converter component configured to generate a digitized detected light signal based on data received from the at least one photodiode;
a processor configured to execute instructions to process the digitized detected light signals;
a protrusion surrounding the at least two light emitting components and the at least one photodiode to prevent external light from entering a region bounded by the protrusion; and
at least one filter mounted over the at least one photodiode.

12. The device of claim 11 wherein the at least one filter is a near-infrared cut-off filter.

13. The device of claim 11 wherein the at least two light emitting components each emit light within a range of about 400 nm to about 950 nm.

14. The device of claim 11 wherein the protrusion and the body are co-molded and made of the same light blocking material.

15. The device of claim 11 wherein the light blocking material is a plastic containing carbon black.

16. A material configured to block ambient light for use with an optical device non-invasively perform tissue monitoring, the material comprising:
a first layer of a flexible, high-optical density material; and
a second layer of an elastane,
wherein the optical device comprises at least two light emitting components and at least one photodiode configured to receive reflected light generated from the at least two light emitting components.

17. The material of claim 16, wherein the first layer is selected from Dartex®, neoprene, and combinations thereof.

18. A device operable to non-invasively perform tissue monitoring, the device comprising:
at least two light emitting components having wavelengths in the visible range of the spectrum;
at least one photodiode operable to receive reflected light generated from the at least two light emitting components, wherein the at least one photodiode is spaced apart from the at least two light emitting components;
an analog-to-digital converter component operable to generate a digitized detected light signal based on data received from the at least one photodiode;
a processor operable to execute instructions to process the digitized signal; and
at least one NIR-cut filter mounted over the at least one photodiode.

19. The device of claim 18 wherein at least one of the visible wavelengths is an isobestic wavelength.

20. The device of claim 18 wherein the wavelengths are 440 nm, 505 nm, and 650 nm.
